(19) United States
(12) Patent Application Publication
(10) Pub. No.: US 2011/0159088 A1
(43) Pub. Date: Jun. 30, 2011

(54) ORAL PHARMACEUTICAL FOR BASED ON AT LEAST ONE ACTIVE PRINCIPLE WHOSE SOLUBILITY VARIES AS A FUNCTION OF THE GASTRIC pH CONDITIONS

(75) Inventors: Florence Guimberteau, Montussan (FR); Gérard Soula, Meyzieu (FR)

(73) Assignee: Flamel Technologies, S.A., Venissieux Cedex (FR)

(21) Appl. No.: 11/920,741

(22) PCT Filed: May 24, 2006
(86) PCT No.: PCT/EP2006/062609

§ 371 Date: Feb. 23, 2009

(30) Foreign Application Priority Data
May 24, 2005 (FR) 0551345

(51) Int. Cl. A61K 9/52; A61K 9/00; A61K 9/28; A61K 31/4178; A61K 31/41; A61K 31/522; A61P 3/00; A61P 9/00; A61P 31/00; A61P 35/00; A61P 25/00

(52) U.S. Cl. 424/456; 424/400; 424/468; 514/397; 514/381; 514/263.38

(57) ABSTRACT

The field of the present invention is that of oral pharmaceutical forms of at least one active principle AP whose solubility varies greatly as a function of the gastric pH, and also treatments and administration methods relating thereto.

The invention relates to the use, in an oral pharmaceutical form comprising AP, of a coating or of a matrix including the said AP and allowing the controlled release of the said AP, in order for this form administered orally to a sample of individuals to lead, irrespective of the fed or fasted state of the individuals, to a reduction in the inter- and/or intra-individual standard deviation of the Cmax, which makes it possible to ensure lower variability of the efficacy and of the therapeutic safety of the pharmaceutical form, compared with an immediate-release AP pharmaceutical form administered to this same sample of individuals, at the same dose.

### FIELD OF THE INVENTION

The field of the present invention is that of oral pharmaceutical forms of active principle(s) -AP- whose solubility varies greatly as a function of the gastric pH conditions, and also treatments and administration methods relating thereto.

For the purposes of the present description, the abbreviation “AP” denotes either a single active principle or a mixture of several active principles, with the exclusion of losartan. In addition, the abbreviation “AP” denotes the AP per se and/or at least one salt, ester or other pharmaceutically acceptable form thereof, including metabolites thereof.

### GENERAL FEATURES

### Problematics

The assurance of quality and reproducibility of a treatment is a major requirement for any pharmaceutical form, and especially for oral AP forms.

However, it may arise that certain oral AP pharmaceutical forms do not satisfy this requirement and thus, for the same therapeutic form administered orally at the same dose, certain patients benefit from an adequate and effective therapeutic protection whereas certain others are incorrectly treated and/or, even more seriously, are victims of hazardous side effects.

Such oral AP pharmaceutical forms lead to erratic plasmatic profiles and do not guarantee a therapeutic treatment that is homogeneous, effective and tolerable for all patients.

These serious drawbacks are observed for immediate-release oral pharmaceutical forms (IRF) that may be administered one or more times a day.

In particular, it has been observed that the plasmatic concentration profiles obtained after administration of oral IR pharmaceutical forms of AP result, for 1 to 25% of patients, in a high and often early plasmatic concentration peak, whereas, for the majority of patients, the plasmatic concentration peak is smaller and occurs later.

Variations in pharmacokinetic profiles may be observed in the same patient, depending on his general state, depending on whether the medicinal product is taken in the fed or fasted state, or alternatively depending on the time at which it is taken.

This high variability with premature and massive release of AP may have serious consequences, especially when the AP is an antihypertensive agent.

Firstly, the patients for whom the concentration peak is of very high amplitude may suffer from serious side effects, for example hypotension when the AP is an antihypertensive agent, or from hypoglycaemia when the AP is a hypoglycaemiant.

Secondly, the early decrease in the plasmatic concentration after the peak is reflected by a very low concentration of AP, at the end of the period between two administrations. Thus, after having been subjected to an overconcentration of AP corresponding to the peak, these patients are insufficiently treated at the end of the period between two administrations.

Finally, this high variability leads the medical practitioner to limit the prescribed doses for all patients and, as a result, certain patients may be incorrectly treated.

It would therefore be advantageous to have available oral AP pharmaceutical forms that make it possible to avoid, in particular for the forms that may be administered once a day, an erratic behaviour of the plasmatic concentration profile. In other words, it would be advantageous for the pharmaceutical form to lead to a homogeneous population of plasmatic concentration profiles without massive and/or early and/or rapid release of the AP. This objective is thus different from the search for a form for sustained release of AP.

### PRIOR ART

Monolithic oral pharmaceutical forms and multimicroparticulate oral pharmaceutical forms are known.

### Monolithic Forms

Patent application WO-A-98/24411 describes a therapeutic treatment method using buspirone, which consists in orally administering an immediate-release dosage form (e.g. tablet or gel capsule) comprising both buspirone and a sufficient amount of nefazodone, so as to increase the bioavailability of the buspirone and to reduce its elimination, the formation of metabolite and also the variability of the pharmacokinetic parameters. This combination of nefazodone with buspirone is supposed to overcome the problem of the high level of variability of the pharmacokinetic parameters, observed for the controlled/sustained-release formulations of buspirone disclosed in U.S. Pat. No. 5,431,922 (cf page 3, lines 7 to 16 of WO-A-98/24411).

U.S. Pat. No. 6,248,359 discloses a multitablet system for treating urinary incontinence using oxybutynine. This system comprises a 1^{st} tablet that releases the oxybutynine over a short period of time (e.g. less than 6 hours) and a 2^{nd} tablet that releases oxybutynine over an extended period of time (18 to 24 hours). This system is presented as being able to compensate for the inter-individual variability, in response to treatment with oxybutynine. These tablets each comprise, for example, an oxybutynine core and several coats. Oxybutynine is of high solubility in acidic medium. This solubility does not vary greatly as a function of the gastric pH conditions.

### Multimicroparticulate Forms

Patent application PCT WO-A-96/11675 describes microcapsules for the oral administration of medicinal and/or nutritional active principles (AP), which are less than or equal to 1000 μm in size. These microcapsules consist of particles coated with a coating material, which itself consists of a mixture of a film-forming polymer (ethylcellulose), a hydrophobic plasticizer (castor oil), a surfactant and/or lubricant (magnesium stearate) and a nitrogenous polymer (polyvinylpyrrolidone: PVP). These microcapsules are also characterized by their ability to remain for a long time (at least 5 hours) in the small intestine and to allow, during this residence time, the absorption of the AP over a period longer than the natural transit time in the small intestine.

Patent application PCT WO-A-03/030878 describes a system for the delayed, controlled and definite release of AP, characterized by a two-fold mechanism of initiation of release of the AP: 1) “time-dependent” release initiated after a controlled time in the stomach, without change of pH, and 2) “pH-dependent” release initiated by a rise in pH, when the dosage form enters the intestine. These microcapsules with a diameter of between 200 and 600 microns are characterized by a coating film based on a hydrophilic polymer A of Eudragit® L type combined with a hydrophobic compound B, such as a plant wax (Lubritab®) with a melting point of between 40 and 90° C., the ratio B/A being between 0.2 and 1.5.

Patent application US-A-2005/0059667 relates to a sustained-release formulation of ranolazine in the form of tablets obtained by compressing granules. Each granule is, for example, based on ranolazine, at least one partially pH-dependent binder such as the methacrylic acid copolymer (Eudragit® L 100-55), microcrystalline cellulose, polyvinylpyrrolidone, and copolymer of methyl methacrylate and of ethyl methacrylate (Eudragit® NE 30D). The granules are mixed with magnesium stearate and sodium croscarmellose, for the compression. The tablets may be coated with an enteric coating or with a coating based on Opadry®. The partially pH-dependent binder such as methacrylic acid copolymer is essentially insoluble at a pH below 5.5 and soluble at a pH above 5.5. This formulation may be administered orally twice a day and allows control of the rate of dissolution of the ranolazine and maintains the plasmatic concentration between 550 and 7500 ngbase/ml. It is indicated in the said patent that the problem of ranolazine is that of having high solubility at low gastric pH values. Ranolazine has a short plasmatic half-life. This high solubility at low gastric pH values results in rapid absorption and elimination of ranolazine and also in undesirable plasmatic fluctuations and a short duration of action. As a result, the oral administrations must be frequent in order to obtain an adequate treatment. The invention according to US-A-2005/0 059 667 claims to overcome this by recommending the administration once or twice a day of the above-described formulation, which is supposed to produce plasmatic concentrations effective for treating angina pectoris.

U.S. Pat. No. 5,576,533 describes oral pharmaceutical forms comprising a plurality of controlled-release microgranules of furosemide, individually coated with a mucoadhesive membrane. These pharmaceutical forms are presented as reducing the inter- and/or intra-individual variability.

None of these known oral pharmaceutical forms is presented as offering an assurance in terms of inter- and/or intra-individual reproducibility of the plasmatic concentration profile with elimination of the risk of premature and massive release and therapeutic cover throughout the time interval between two dosage intakes.

The said oral forms can thus be improved.

To the Inventors' knowledge, the prior art is thus lacking in technical proposals capable of providing an initial solution to this problem of oral pharmaceutical forms leading to erratic plasmatic profiles.

### OBJECTIVES

On the strength of these observations, the Inventors set themselves the following objectives.

An essential objective of the invention is to provide an oral pharmaceutical form of AP that is used such that it gives access to a more uniform and more reproducible quality of treatment from one patient to another and/or for a patient, compared with what is proposed in the prior art.

Another essential objective of the present invention is to propose a means for reducing the inter- and/or intra-individual standard deviation of the maximum concentration Cmax and/or of the moment of passage to the concentration maximum, Tmax, of the plasmatic concentration profile.

Another essential objective of the invention is to provide an oral pharmaceutical form of AP that allows a reduction in the inter- and/or intra-individual variability of the plasmatic concentration profiles of the known oral pharmaceutical forms of AP, in order especially to avoid the appearance of a “rapid” at-risk population, for which the plasmatic profile has a high and early concentration peak.

Another essential objective of the invention is to provide an oral pharmaceutical form of AP that offers an assurance in terms of therapeutic safety: elimination of the risk for certain patients of massive and/or rapid release of the AP and therapeutic cover throughout the time interval between two dosage intakes.

Another essential objective of the invention is to provide an oral pharmaceutical form of AP that guards the patients against any risk of plasmatic overconcentration of the AP and thus protects them against any medication-related accident.

Another essential objective of the invention is to propose a means for reducing the peak/trough ratio of the plasmatic concentrations of AP.

Another essential objective of the invention is to provide an oral pharmaceutical form of AP that can reduce the inter- and/or intra-individual variability of the plasmatic concentration profiles of the known oral pharmaceutical forms of AP, in order especially to avoid the appearance of two population profiles of plasmatic concentrations: one population Pr at risk of “rapid” profiles and one population P1 of “slow” profiles.

Another essential objective of the present invention is to propose a means for reducing, or even eliminating, the rapid population Pr.

Another essential objective of the invention is to propose, for the oral pharmaceutical forms, a novel use of the means for controlling the release of the AP (coating or matrix containing the AP) so as to satisfy at least one of the above objectives, and in particular to reduce the inter- and/or intra-individual variability of the plasmatic profiles.

Another essential objective of the invention is to propose a novel use of oral pharmaceutical forms comprising means for controlling the release of the AP, of the coating or matrix type containing the AP, so as to satisfy at least one of the above objectives.

Another essential objective of the invention is to propose, for the oral pharmaceutical forms, a novel use of the means for controlling the release of the AP (coating or matrix containing the AP), in order to reduce the inter- and/or intra-individual variability of the plasmatic concentration profiles, and especially to reduce the inter- and/or intra-individual standard deviation of the maximum plasmatic concentration after administration.

Another essential objective of the invention is to propose a novel use of oral pharmaceutical forms comprising means for controlling the release of the AP of the coating or matrix type containing the AP, in order to reduce the inter- and/or intra-individual variability of the plasmatic concentration profiles and especially to reduce the inter- and/or intra-individual standard deviation of the maximum plasmatic concentration after administration.

Another essential objective of the invention is to provide a therapeutic method that consists in using an oral pharmaceutical form that satisfies at least one of the above therapeutic objectives.

All the improvements targeted above are in reference to an oral pharmaceutical form with immediate release of AP.

### BRIEF DESCRIPTION OF THE INVENTION

In this context, it is to the Applicant's credit:
to have formulated the hypothesis that the non-reproducibility of the quality of treatment from one patient to another and/or in the same patient could be linked to the large variation of solubility in the pH range present in the stomach. This variability of solubility, which conditions the rate of bioabsorption, thus constitutes an important factor in the variability of the pharmacokinetics of the medicament;
to have realized that this variability in gastric pH depends on various uncontrollable parameters, especially: fed or fasted state, time of the dosage intake, inter- and/or intra-individual variability, action of a medicament having an influence on these gastrointestinal conditions, etc.;
and, finally, to have imagined a technical solution for limiting or even eliminating this dependence, this solution consisting in recommending the use of a coating or a matrix containing the AP, which is capable of reducing or even eliminating the rapid plasmatic profile population Pr and of avoiding the premature and/or massive and/or rapid release of the AP irrespective of the gastric acidity, which is of a nature to reduce the inter- and/or intra-individual variability of the plasmatic concentration profiles. In so doing, firstly, the therapeutic safety is improved by preventing the deleterious effects of the oral administration of AP for a certain population of patients, and, secondly, the therapeutic efficacy is promoted.

Thus, the present invention achieves the above objectives, among others, by proposing:
the use
   in an oral pharmaceutical form comprising at least one AP whose solubility varies by a factor of at least 3, preferably at least 10 and even more preferentially at least 30 under gastric pH conditions of between 1.0 and 5.5 and preferably between 1.5 and 5.0, of a coating or of a matrix including the said AP and allowing the controlled release of the said AP,
   in order for this form administered orally to a sample of human individuals to lead, irrespective of the fed or fasted state of the individuals, to a reduction in the inter- and/or intra-individual standard deviation of the Cmax and/or of the Tmax,
   which makes it possible to ensure a lower variability of the efficacy and of the therapeutic safety of the pharmaceutical form,
   relative to an immediate-release AP pharmaceutical form administered to this same sample of individuals, at the same dose;
and/or the use
   of AP contained in a coated form or in a matrix allowing the controlled release of this AP, whose solubility varies by a factor of at least 3, preferably at least 10 and even more preferentially at least 30 under gastric pH conditions of between 1.0 and 5.5 and preferably between 1.5 and 5.0,
   for the manufacture of an oral pharmaceutical form which, after oral administration to a sample of human individuals, leads, irrespective of the fed or fasted state of the individuals, to a reduction in the inter- and/or intra-individual standard deviation of the Cmax and/or of the Tmax,
   which makes it possible to ensure a lower variability of the efficacy and of the therapeutic safety of the pharmaceutical form,
   relative to an immediate-release AP pharmaceutical form administered to this same sample of individuals, at the same dose.

The invention thus relates to pharmaceutical forms containing at least one AP whose solubility varies greatly as a function of the gastric pH.

The invention is thus defined by means of a reference clinical test in which the pharmaceutical form is administered orally to a sample of human individuals, under experimental conditions which may be those given in the examples below. This clinical test defines the invention by the pharmacokinetic properties specifically obtained under the test conditions. However, the invention is not limited to an implementation under the conditions of this reference clinical test.

The use according to the invention makes it possible to reduce, or even eliminate, the erratic nature of the plasmatic concentration profiles from one individual to another and, in so doing, to avoid, firstly, the premature release of the AP and thus a plasmatic overconcentration with the side effects thereby entailed, and, secondly, any possible lack of therapeutic cover between two dosage intakes.

Thus, the technical function exploited and highlighted in accordance with the invention is not the extension of the release time, but rather the reduction of the variability of the treatment that may be detrimental to the patient. Thus, the invention makes it possible to ensure better efficacy and greater therapeutic safety.

The gastric pH is a value that is intrinsically variable within a pH range of from pH 1.0 to pH 5.5. This variation is observed for the same individual especially according to the fed or fasted state, and from one individual to another. In addition, certain patients may be treated with medicaments that modify the gastric pH. This is the case, for example, with proton pump inhibitors (e.g. omeprazole) or antiacids.

The Applicant has, to its credit, observed that an AP whose solubility depends greatly on the gastric pH leads to erratic plasmatic concentration profiles from one patient to another and/or in the same patient.

Without the Applicant being able to offer a full explanation for this phenomenon, it may be suggested that this variability in the plasmatic concentration profile follows from the variation in the solubility of the AP as a function of the gastric pH. Specifically, in order to be absorbed, the AP must first be dissolved. This dissolution step thus depends greatly on the gastric pH. Thus, for the same dose of AP, and according to the gastric pH of the patient, the AP dissolves fully and quickly or, on the contrary, does not dissolve in the stomach, depending on the patient or, in the same patient, depending on the administration conditions.

It is thus appreciated that an AP whose solubility depends greatly on the gastric pH, the dissolution, and therefore ultimately the plasmatic concentration profile, undergoes large variations from one individual to another and, for the same individual, from one day to another.

Thus, an immediate-release form (IRF, cf. definition below) of AP, administered at a dose of 100 mg to a sample of 20 individuals, leads to a Cmax varying, from one individual to another, by a factor of greater than 10 (70 to 800 ng/ml).

This erratic nature of the plasmatic concentration profiles may be reflected by the appearance of two populations of profiles: the rapid population and the slow population.

The profiles of the rapid population are those for which a high plasmatic peak is observed very early.

The profiles of the slow population are those for which a high plasmatic peak is not observed very early.

For the rapid population, the premature release of the AP has three very detrimental consequences:
(a) the patients subjected to an early plasmatic overconcentration of AP are potentially subjected to hazardous side effects, such as hypotension or hypoglycaemia;
(b) the existence of these risks leads to the prescribed doses being limited, which may deprive certain patients of an adequate treatment;
(c) for the rapid profiles, the plasmatic concentration is very low at the end of the time interval between two administrations. The therapeutic cover of these patients is consequently insufficient.

Thus, one of the essential elements of the invention consists in using or in proposing the use, for therapeutic purposes, of an oral pharmaceutical form comprising AP contained in a coated form or in a matrix designed to govern the controlled release of the AP, such that this pharmaceutical form administered orally to a sample of human individuals leads, irrespective of the fed or fasted state of the individuals, to a reduction in the inter- and/or intra-individual standard deviation of the Cmax, which makes it possible to ensure smaller variability of the efficacy and of the therapeutic safety of the pharmaceutical form, compared with an immediate-release AP pharmaceutical form administered to this same sample of individuals, at the same dose.

The use of the abovementioned coating or of the abovementioned matrix to manufacture such an oral pharmaceutical form is also targeted by the invention.

The oral pharmaceutical form to which the abovementioned uses relates is also a fully fledged subject of the present invention.

It is thus to the Applicant's credit to have discovered that coating the AP with a controlled-release membrane or including the AP in a controlled-release matrix makes it possible to eliminate or reduce the erratic nature of the AP release profiles from one individual to another.

This invention appears particularly important for optimizing the use of the AP, which may, by itself, be administered once a day, but which suffers from this erratic behaviour of the plasmatic profiles. Thus, the aim of the invention is not mainly the extension of the release time, but especially the reduction of the variability of the treatment that is prejudicial to the patient. Thus, the invention makes it possible to ensure better efficacy and therapeutic safety.

In summary, the Applicant's inventive merit is based essentially on the fact that it has clearly identified and posed the problem of the variability of the solubility of the AP depending on the gastric pH and of the variability of this gastric pH. Starting from these intangible factors, the Applicant has proposed a novel and inventive use of known general means to limit the influence of these factors. These means are the coating membrane or the matrix containing the AP. They make it possible to avoid its rapid and early release in the stomach, even in the case of patients whose gastric pH is such that the solubility of the AP is high.

### DEFINITIONS

For the purposes of the present invention, “an AP whose solubility varies greatly as a function of the gastric pH” is an AP whose solubility varies by a factor of at least 3, preferably at least 10 and even more preferentially at least 30 under gastric pH conditions of between 1.0 and 5.5 and preferably between 1.5 and 5.0.

This solubility is measured in an aqueous medium at 37° C., maintained at the pH under consideration.

In the present specification, the term “reservoir” pharmaceutical form denotes a form in which at least a fraction of the AP is released by crossing a continuous membrane (or film) which controls its diffusion outwards. Non-limiting examples that may be mentioned include microparticles containing AP and individually coated with a membrane or tablets coated with a membrane that controls the diffusion of the AP.

In the present specification, the term “matrix” pharmaceutical form denotes a pharmaceutical form in which the AP is dispersed in a solid continuous phase (the matrix) consisting of pharmaceutically acceptable excipients, this phase not being coated with a continuous membrane (or film) that controls the diffusion of the AP.

In the present specification, the term “immediate release” denotes the release by an Immediate-Release Form (IRF) of the majority of the AP in a relatively short time, for example:
at least 70% of the AP is released in vivo in one hour, and preferably in thirty minutes, after oral ingestion;
or at least 70% of the AP is released in 1 hour, and preferably in thirty minutes, at any pH of between 1.4 and 6.8 in an in vitro dissolution test.

All the in vitro dissolution profiles under consideration in the present specification are produced according to the indications of the European Pharmacopoeia 4^{th} edition entitled: “Test of dissolution of solid oral forms”: type II dissolutest performed under SINK conditions at 37° C. and stirred at 100 rpm.

In the present specification, the term “controlled-release form” denotes a form in which at least a fraction of the AP is released at a rate less than or equal to the rate of an immediate-release form. This fraction may be, for example, between 1% and 100%, preferably between 10% and 100% and even more preferentially between 30% and 100%. Thus, such a controlled-release formulation may comprise, for example, an immediate-release phase and a slow- or delayed-release phase. Controlled-release formulations are well known in this field; see, for example, Remington: The science and practice of pharmacy, 19th edition, Mack publishing Co. Pennsylvania, USA. The controlled release may especially be a sustained and/or delayed release.

The pharmacokinetic parameters under consideration in the present invention are defined in the following manner. After oral administration of the pharmaceutical form to a sample of N human individuals, the individual plasmatic concentration profile is measured on each of the patients, from which the conventional individual pharmacokinetic parameters are drawn: Tmax, Cmax, C24h:
Tmax is the time at which the plasmatic concentration reaches its maximum, Cmax.
C24h is the plasmatic concentration 24 hours after the administration.

From these individual parameters, a person skilled in the art conventionally calculates the mean values of these parameters and their standard deviations. Further details regarding the discussion of these parameters will be found in the book: Pharmacokinetics and pharmacodynamic Data Analysis 3rd ed., J. Gabrelsson et al., Kristianstads Bocktryckeri AB, Sweden, 2000.

### DETAILED DESCRIPTION OF THE INVENTION

According to a first particular embodiment of the invention, the APs whose solubility varies greatly as a function of the gastric pH are APs for which the dispersion of a dose D in a volume of 100 ml of water at 37° C. does not allow, at equilibrium, total dissolution of the dose D for at least one gastric pH value of between 1 and 5.5.

According to a second particular embodiment of the invention, the APs whose solubility varies greatly as a function of the gastric pH are APs for which the dispersion of a dose D in a volume of 100 ml of water at 37° C., —
does not allow, at equilibrium, total dissolution of the dose D for at least one gastric pH value of between 1 and 5.5;
and allows, at equilibrium, total dissolution of the dose D for at least one other gastric pH value of between 1 and 5.5.

According to a third particular embodiment of the invention, the pharmaceutical form is defined by a number of daily dosage intakes that is identical to that of an immediate-release pharmaceutical form comprising the same AP dose.

This third embodiment, like the first two, illustrates the fact that the aim of the invention is not to extend the AP release time, but to reduce the variability.

The AP dissolution test characterizes the invention, but also makes it possible to select the APs with which the invention is concerned and which are subject to the problem of dependence of the solubility of the AP with respect to the gastric pH.

Advantageously, the APs with which the invention is concerned are those having a solubility that is highly dependent on the gastric pH and are chosen, for example, from the group comprising the following families of AP:

proton pump inhibitors, angiotensin II receptor antagonists [or ARB (Angiotensin Receptor Blocker)], antiulcer agents, antidiabetic agents, anticoagulants, antithrombotic agents, hypolipidaemiants, antiarrhythmic agents, vasodilators, antiangina agents, antihypertensives, vasoprotective agents, fertility promoters, labour inducers and inhibiters, contraceptives, antibiotics, antifungal agents, antiviral agents, anticancer agents, anti-inflammatory agents, analgesics, antiepileptics, antiparkinson agents, neuroleptic agents, hypnotic agents, anxiolytic agents, psychostimulators, antimigraine agents, antidepressants, antitussif agents, antihistamine or antiallergic agents, agents for combating congestive heart insufficiency, angina pectoris, left ventricular hypertrophy, cardiac arrhythmia, myocardial infarction, reflex tachycardia, ischaemic heart disease, atheromatosis, hypertension associated with diabetes mellitus, portal hypertension, vertigo, bradycardia, arterial hypotension, water retention, acute renal insufficiency, orthostatic hypotension and cerebral congestion, and combinations of all the products mentioned above taken within one or more families.

ARBs are preferred, and more particularly the ARBs selected from the subgroup comprising:

Irbesartan, Olmesartan, Eprosartan, Candesartan, Candesartan Cilexetil, Valsartan, Telmisartan, Zolasartin, Tasosartan.

The ARBs may be combined with at least one active co-principle chosen from diuretics (hydrochlorothiazide), beta-blockers, angiotensin-converting enzyme inhibitors, sodium channel blockers, alpha-blockers, alpha-beta-blockers, vasodilators, alpha-antagonists and adrenergic neuronal blockers. For further details regarding these active co-principles for ARBs, reference may be made, for example, to the passage on page 4, line 19-page 4, line 31 of WO-A-03/035039.

As examples of APs other than ARBs, mention may be made of APs whose solubility is greatly dependent on the pH and which belong to the non-limiting set of compounds chosen from the following group of compounds: acetylsalicylic acid, carbamazepine pentoxifylline, prazosine, acyclovir, nifedipine, diltiazem, naproxen, ibuprofen, flurbiprofen, ketoprofen, fenoprofen, indomethacin, diclofenac, fentiazac, oestradiol valerate, metoprolol, sulpiride, captopril, cimetidine, zidovudine, nicardipine, terfenadine, atenolol, salbutamol, carbamazepine, ranitidine, enalapril, simvastatine, fluoxetine, alprazolam, famotidine, ganciclovir, famciclovir, spironolactone, 5-asa, quinidine, perindopril, morphine, pentazocine, paracetamol, omeprazole, lansoprazole, metoclopramide, aminosalicylic acid, nalidixic acid, amoxicillin, amoxicillin and potassium clavulanate, ampicillin, ampicillin and sulbactam, azithromycin, bacampicillin, carbenicillin-indanyl-sodium (and other salts of carbenicillin), capreomycin, cefadroxile, cefazoline, cephalexine, cephalothine, cephapirine, cephacelor, cephprozile, cephadrine, cefamandole, cefonicide, ceforanide, cefuroxime, cefixime, cefoperazone, cefotaxime, cefpodoxime, ceftaxidime, ceftibuten, ceftizoxime, ceftriaxone, cefepime, cefmetazole, cefotetane, cefoxitine, ciprofloxacine, clarithromycin, clindamycin, clofazimine, cloxacillin, co-triamoxazole, cycloserine, dicloxacillin, dirithromycin, erythromycin (and other salts such as erythromycin estolate, ethylsuccinate, gluceptate, lactobionate, stearate), ethambutol-HCl and other salts, ethionamide, fosfomycin, imipenem, isoniazide, levofloxacine, lomefloxacine, loracarbef, methicillin, methenamine, metronidazole, metoclopramide, mezlocillin, nafcillin, nitrofurantoin, norfloxacine, novobiocine, ofloxacine, oxacillin, penicillin, penicillin salts, penicillin complexes, pentamidine, piperacillin, piperacillin and tazobactam, sparfloxacine, sulfacytine, sulfamerazine, sulfamethazine, sulfamethixole, sulfasalazine, sulfisoxazole, sulfapyrizine, sulfadiazine, sulfinethoxazole, sulfapyridine, ticarcillin, ticarcillin and potassium clavulanate, trimethoprime, trimetrexate, troleanomycin, vancomycin and verapamil, and mixtures thereof.

According to one variant, the AP does not comprise diuretics.

The standard deviation reduction factor (f) is defined by the ratio of the standard deviation of the Cmax for the IRF* form to the corresponding standard deviation of the form concerned by the use according to the invention. Preferably, the inter-individual standard deviation reduction factor (f) of the Cmax is such that:

f≧1.05; preferably f≧1.5 and even more preferentially f is between 2.0 and 20.

According to another advantageous arrangement of the invention, the pharmaceutical form may be administered daily by means of one or more dosage units of any type (e.g. tablet, gel capsule, volume unit of powder or of liquid), with the exclusion of systems including several tablets per dosage intake, in which at least one of these tablets is a tablet with rapid release of the active principle (less than six hours) and at least one other of these tablets is a tablet with sustained release of the same active principle (18 to 24 hours).

Advantageously, the coating and the matrix are designed such that they allow the controlled release of the AP, firstly to prevent any premature and/or massive and/or rapid release of the AP and subsequently any deleterious plasmatic overconcentration of AP, and secondly to ensure therapeutic cover between two dosage intakes.

According to one variant, the coating is non-mucoadhesive within the meaning of U.S. Pat. No. 5,571,533.

According to one advantageous characteristic of the invention, the core of the sustained-release AP microparticles is not lipophilic, i.e., for example, it does not contain one or more excipients having an overall HLB of less than 8.

For the purposes of the present invention, the mean peak/trough modulation—PTM—of the plasmatic profile of an AP is defined in the following manner: on each of the individual plasmatic profiles, the individual maximum concentration cmax′ and the concentration cT′, T hours after a single oral administration are measured. The PTM is the arithmetic mean of the individual cmax′/cT′ ratios.

For a product intended to be administered daily to a patient, T is 24 hours after the single administration. If the concentration cT′ (T=24 hours) is less than the detection limit of the assay method used and below the detection limit of the method recommended by the US Pharmacopea and/or known to those skilled in the art, the concentration c24′ used to calculate the PTM will be replaced by the concentration cx′ measured x hours after oral administration, x being the latest time at which a concentration above the detection limit of the used method can be measured. In this case, x is less than 24 hours after single administration. For example x is equal to 18 hours or, failing that, 12 hours.

For a product intended to be administered twice daily to the patient, T is 12 hours in single administration. In this case also, if the concentration cT′ (T=12 hours) is less than the detection limit of the assay method used and below the detection limit of the method recommended by the US Pharmacopea and/or known to those skilled in the art, the concentration c12′ used to calculate the PTM will be replaced by the concentration cx′ measured x hours after oral administration, x being the latest time at which a concentration above the detection limit of the used method may be measured. In this case, x is less than 12 hours after single administration.

In accordance with the use according to the invention, the coating and the matrix of the pharmaceutical form are designed such that the oral administration of this form, to a sample of human individuals, leads to a mean peak/trough modulation (PTM) of the plasmatic concentration profiles of the AP, less than the mean peak/trough modulation of the AP for the same sample of individuals who received the same dose of an immediate-release AP form.

For the purposes of the invention, the reduction in the peak/trough modulation of the plasmatic concentration profiles is given, for example, by the peak/trough modulation reduction factor g. The factor g is defined by the ratio of the peak/trough modulation of the IRF* form to the peak/trough modulation of the form concerned by the use according to the invention.

Preferably, the peak/trough modulation reduction factor g is such that:

g≧1.05; preferably g≧1.5 and even more preferentially g is between 2.5 and 20.

In accordance with the use according to the invention, the coating and the matrix of the pharmaceutical form are designed such that the oral administration of this form, to a sample of human individuals, leads to a variability of the peak/trough modulation of the plasmatic profiles of the AP, less than the variability of the peak/trough modulation of the AP for the same sample of individuals who received the same dose of an immediate-release AP form.

For the purposes of the invention, the reduction in the variability of the peak/trough modulation of the plasmatic concentration profiles is given, for example, by the factor g′ for reduction of the standard deviation of the peak/trough modulation. The factor g′ is defined by the ratio of the standard deviation of the peak/trough modulation of the IRF* form to the standard deviation of the peak/trough modulation of the form concerned by the use according to the invention.

Preferably, the factor g′ for reduction of the standard deviation of the peak/trough modulation is such that: g′≧1.05; preferably g′≧1.5 and even more preferentially g′ is between 2.5 and 20.

The plasmatic profiles obtained are more uniform. Their inter- and/or intra-individual variability is reduced.

In a noteworthy manner, the invention also proposes:
the use, in an oral pharmaceutical form comprising AP, of a coating or of a matrix including the said AP allowing the controlled release of AP, such that this pharmaceutical form administered orally, to a sample of human individuals (for example in the fed state), leads to a decrease in the number or to the disappearance of the individual plasmatic profiles having a Tmax of less than or equal to one hour and preferably less than or equal to 1.5 hours, to the benefit of the individual plasmatic profiles having a Tmax of greater than one hour and preferably greater than 1.5 hours, which makes it possible to ensure a lower variability of the efficacy and of the therapeutic safety of the pharmaceutical form, relative to an immediate-release AP pharmaceutical form administered to this same sample of individuals (for example in the fed state), at the same dose;
or the use of AP contained in a coated form or in a matrix allowing the controlled release of AP, for the manufacture of a pharmaceutical form which, after oral administration to a sample of human individuals (for example in the fed state) leads to a decrease in the number or to the disappearance of the individual plasmatic profiles having a Tmax of less than or equal to one hour and preferably less than or equal to 1.5 hours, to the benefit of the individual plasmatic profiles having a Tmax of greater than one hour and preferably greater than 1.5 hours, which makes it possible to ensure a lower variability of the efficacy and of the therapeutic safety of the pharmaceutical form, compared with an immediate-release AP pharmaceutical form administered to this same sample of individuals (for example in the fed state), at the same dose.

Comparison of the controlled-release AP form used according to the invention and of the immediate-release form (IRF*), and in particular of the pharmacokinetic parameters of their standard deviation, is performed under the same conditions and at the same dose of AP, on a number of volunteers, for example greater than or equal to fifteen and preferably greater than or equal to 20.

Even more specifically, and still by way of example, the comparison may be made by means of a reference clinical test according to which a medicament is administered orally to a sample of N human individuals, preferably N≧20 or 30 individuals. The individual plasmatic concentration profile is then measured on each of the volunteers, from which the individual pharmacokinetic parameters are drawn, such as the time Tmax at which the plasmatic concentration reaches its maximum and the value of this maximum concentration Cmax. From these individual parameters, a person skilled in the art conventionally calculates the mean values of these parameters and their standard deviations. Further details regarding the discussion of these parameters will be found in the book: Pharmacokinetics and pharmacodynamics Data Analysis 3rd ed., J. Gabrelsson et al., Kristianstads Bocktryckeri AB, Sweden, 2000.

The experimental conditions of the reference clinical test may be, for example, as follows: administration of the form (gel capsule, tablet or suspension) once a day, at a given dose, after breakfast, to 20 healthy volunteers in the course of a crossed-test study. The plasmatic concentrations of AP are measured, for example, at times: 0-0.25-0.5-0.75-1-1.5-2-3-4-6-8-10-12-16-18-20-24-36-48 hours post-administration. This clinical test defines the invention by the pharmacokinetic properties specifically obtained under the test conditions. However, the invention is not limited to an implementation under the conditions of this reference clinical test.

According to one variant, the invention is also directed towards:
the use, in an oral pharmaceutical form comprising AP, of a coating or of a matrix including the said AP, to reduce the variability of the plasmatic profiles during the administration of this pharmaceutical form to a sample of individuals, compared with an immediate-release AP pharmaceutical form IRF* administered to this same sample of individuals, at the same dose, which makes it possible to ensure lower variability of the efficacy and of the therapeutic safety of the pharmaceutical form, compared with a pharmaceutical form with immediate release IRF* of AP administered to this same sample of individuals, at the same dose;
or the use of AP contained in a coated form or in a matrix, for the manufacture of a pharmaceutical form leading to a reduction in the variability of the plasmatic concentration profiles during the administration of this pharmaceutical form to a sample of individuals, compared with a pharmaceutical form with immediate release IRF* of AP administered to this same sample of individuals, at the same dose, which makes it possible to ensure lower variability of the efficacy and of the therapeutic safety of the pharmaceutical form, compared with an immediate-release AP pharmaceutical form administered to this same sample of individuals, at the same dose.

The pharmaceutical form targeted in the use according to the invention may contain AP in the form of microunits, which may especially be:
microparticles individually consisting of a core that comprises AP and that is coated with at least one coating allowing the controlled release of the AP (also referred to hereinbelow as coated microparticles);
and/or microgranules individually consisting of a matrix that includes AP and that allows the controlled release of the AP (also referred to hereinbelow as matrix microgranules);
and/or immediate-release AP microgranules.

The oral pharmaceutical form targeted in the use according to the invention may be any of the forms known to those skilled in the art, i.e. especially gel capsules, sachets, suspensions containing microunits of AP or tablets.

These tablets may be
(i) either tablets containing micro-units of AP,
(ii) or tablets free of microparticles individually consisting of a core comprising AP and being coated with at least one coating allowing the controlled release of the AP, and/or free of microgranules individually consisting of a matrix including AP and allowing the controlled release of the AP.

These tablets (ii) may be matrix tablets or individually coated tablets.

The microunits of AP (controlled-release coated microparticles and/or matrix microgranules or immediate-release microgranules) preferably have a mean diameter (Dm in μm) of less than 1000, preferably between 50 and 800 and even more preferentially between 50 and 500.

Advantageously, the oral pharmaceutical form intended in the use according to the invention makes it possible to obtain, after a dosage intake, a plasmatic profile defined as follows:

Cmax/C24h≦Cmax*/C24h*

preferably 1.5×Cmax/C24h≦Cmax*/C24h*

and even more preferentially 2.0×Cmax/C24h≦Cmax*/C24h*

with
C24h representing the mean plasmatic concentration of AP 24 hours after intake,
C24h* representing the mean plasmatic concentration of AP obtained under the same conditions as C24h, with a reference immediate-release oral pharmaceutical form, containing the same dose of AP.
Cmax representing the mean maximum plasmatic concentration of AP after intake,
Cmax* representing the mean maximum plasmatic concentration of AP obtained under the same conditions as Cmax, with a reference immediate-release oral pharmaceutical form, containing the same dose of AP.

In accordance with a first embodiment, the oral pharmaceutical form comprises coated or matrix microparticles and has an in vitro dissolution profile such that:

the time t (70%) after the administration and after which 70% of the AP is released is between 1 and 24 hours, preferably between 2 and 12 hours and even more preferentially between 2 and 8 hours.

According to one advantageous characteristic of this first embodiment, for any value of the time t between 2 hours and t (70%), preferably between 1 hour and t (70%), the percentage of dissolved AP is greater than or equal to 35 t/t (70%).

The composition of the individual coating or of the individual matrix of the microparticles according to the first embodiment advantageously corresponds to one of the following two families A and B:

### Family A

A-1—at least one film-forming polymer (P1) that is insoluble in the fluids of the gastrointestinal tract, present (weight % of solids) in a proportion of from 50 to 90 and preferably 50 to 80 relative to the total mass of the coating composition and especially comprising at least one water-insoluble cellulose derivative;
A-2—at least one nitrogenous polymer (P2) present (weight % of solids) in a proportion of from 2 to 25 and preferably 5 to 15 by weight of solids relative to the total mass of the coating composition and consisting of at least one polyacrylamide and/or one poly-N-vinylamide and/or one poly-N-vinyllactam;
A-3—at least one plasticizer present (weight % of solids) in a proportion of from 2 to 20 and preferably from 4 to 15 by weight of solids relative to the total mass of the coating composition and consisting of at least one of the following compounds: glycerol esters, phthalates, citrates, sebacates, cetyl alcohol esters, castor oil;
A-4—at least one surfactant and/or lubricant, present (weight % of solids) in a proportion of from 2 to 20 and preferably from 4 to 15 by weight of solids relative to the total mass of the coating composition and chosen from anionic surfactants and/or from nonionic surfactants and/or from lubricants; the said agent possibly comprising only one or a mixture of the abovementioned products.

Examples of compounds A-1, A-2, A-3 and A-4 are cited below:

A-1: ethylcellulose and/or cellulose acetate;
A-2: polyacrylamide and/or polyvinylpyrrolidone;
A-3: castor oil;
A-4: alkali metal or alkaline-earth metal salt of fatty acids, stearic acid and/or oleic acid being preferred, polyoxyethylenated ester of sorbitan, polyoxyethylenated castor oil derivatives, stearates, preferably calcium, magnesium, aluminium or zinc stearate.

### Family B:

B1—at least one film-forming polymer that is insoluble in the fluids of the gastrointestinal tract,
B2—at least one water-soluble polymer,
B3—at least one plasticizer,
B4—and optionally at least one surfactant/lubricant preferably consisting of at least one anionic surfactant and/or at least one nonionic surfactant.

Examples of compounds B1, B2, B3 and B4 are cited below:

### B1

water-insoluble cellulose derivatives, ethylcellulose and/or cellulose acetate being particularly preferred,
acrylic polymers,
polyvinyl acetates,
and mixtures thereof.

### B2:

water-soluble cellulose derivatives,
polyacrylamides,
poly-N-vinylamides,
poly-N-vinyllactams,
polyvinyl alcohols (PVA),
polyoxyethylenes (POE),
polyvinylpyrrolidones (PVP) (the latter being preferred),
and mixtures thereof;

### B3:

glycerol and esters thereof, preferably in the following subgroup: acetylated glycerides, glyceryl monostearate, glyceryl triacetate, glyceryl tributyrate,
phthalates, preferably in the following subgroup: dibutyl phthalate, diethyl phthalate, dimethyl phthalate, dioctyl phthalate,
citrates, preferably in the following subgroup: acetyl tributyl citrate, acetyl triethyl citrate, tributyl citrate, triethyl citrate,
sebacates, preferably in the following subgroup: diethyl sebacate, dibutyl sebacate,
adipates,
azelates,
benzoates,
plant oils,
fumarates, preferably diethyl fumarate,
malates, preferably diethyl malate,
oxalates, preferably diethyl oxalate,
succinates, preferably dibutyl succinate,
butyrates,
cetyl alcohol esters,
salicylic acid,
triacetin,
malonates, preferably diethyl malonate,
castor oil (this being particularly preferred),
and mixtures thereof;

### B4:

alkali metal or alkaline-earth metal salts of fatty acids, stearic acid and/or oleic acid being preferred,
polyoxyethylenated oils, preferably polyoxyethylenated hydrogenated castor oil,
polyoxyethylene-polyoxypropylene copolymers,
polyoxyethylenated sorbitan esters,
polyoxyethylenated castor oil derivatives,
stearates, preferably calcium, magnesium, aluminium or zinc stearate,
stearylfumarates, preferably sodium stearylfumarate,
glyceryl behenate,
and mixtures thereof.

Preferably, the coating consists of a single coat, the mass of which represents from 1% to 50% by weight and preferably from 5% to 40% by weight of the total mass of the microparticles.

Other details and examples of compositions and processes for obtaining microparticles according to the first embodiment according to the invention are given in WO-A-03/084 518, the content of which is incorporated into the present specification by reference.

For further qualitative and quantitative details regarding the coating compositions of families A1 & A2, reference will be made, respectively, to European patent EP-B-0 709 087 and to patent application WO-A-2004/010 984, the content of which is incorporated into the present specification by reference.

In accordance with a second embodiment, the oral pharmaceutical form firstly comprises coated microparticles and/or matrix microparticles, and secondly is such that:
the release of the AP is governed by two different initiation mechanisms, one being based on a pH variation and the other allowing the release of the AP after a predetermined residence time in the stomach;
at a constant pH of 1.4, the dissolution profile comprises a lag phase lasting less than or equal to 7 hours, preferably less than or equal to 5 hours and even more preferentially between 1 and 5 hours,
and the passage from pH 1.4 to pH 7.0 leads to a release phase starting without any lag time.

Advantageously, the oral pharmaceutical form according to this second mode has a dissolution profile, measured in an in vitro dissolution test, as indicated below:
less than 20% of the AP is released after 2 hours at pH 1.4;
at least 50% of the AP is released after 16 hours at pH 1.4.

According to one preferred characteristic, the oral pharmaceutical form according to this second mode comprises controlled-release AP microparticles, the initiating pH value of which is between 5.0 and 7.0 inclusive.

According to another preferred characteristic, the oral pharmaceutical form according to this second mode comprises controlled-release AP microparticles the initiating pH value of which is between 6.0 inclusive and 6.5 inclusive.

Advantageously, the controlled-release AP microparticles according to the second embodiment have the following specific features:
the coating or the matrix allowing the controlled release of the AP comprises a composite material
   comprising:
      at least one hydrophilic polymer I bearing groups that are ionized at neutral pH,
      at least one hydrophobic compound II;
   representing a mass fraction (weight % relative to the total mass of the microparticles)≦40; and
their mean diameter is less than 1000 μm, preferably between 50 and 800 μm and even more preferentially between 50 and 500 μm.

According to another advantageous characteristic, the composite material I-II of the coating or of the matrix allowing the controlled release of AP is such that:
the weight ratio II/I is between 0.2 and 1.5 and preferably between 0.5 and 1.0,
and the hydrophobic compound II is selected from products which are crystalline in the solid form and which have a melting point T_{fII}≧40° C., preferably T_{fII}≧50° C. and even more preferentially 40° C.≦T_{fII}≦90° C.

According to one preferred embodiment, the hydrophilic polymer I is chosen from:
I.a copolymers of (meth)acrylic acid and of an alkyl ester of (meth)acrylic acid, and mixtures thereof;
I.b cellulose derivatives, preferably cellulose acetates, cellulose phthalates, cellulose succinates, and mixtures thereof, and even more preferentially hydroxypropylmethylcellulose phthalates, hydroxypropylmethylcellulose acetates and hydroxypropylmethylcellulose succinates, and mixtures thereof;
and mixtures thereof.

The polymers I that are even more preferred are copolymers of (meth)acrylic acids and of alkyl esters (e.g. C1-C6 alkyl) of (meth)acrylic acid. These copolymers are, for example, of the type of sold by the company Röhm Pharma Polymers under the registered trade marks Eudragit®, of the series L and S (for instance Eudragit® L100, S100, L30 D-55 and L100-55). These copolymers are anionic enteric copolymers that are soluble in aqueous medium at pH values above those encountered in the stomach.

Still according to the preferred embodiment, compound II is chosen from the following group of products:
II.a plant waxes taken alone or as mutual mixtures;
II.b hydrogenated plant oils taken alone or as a mutual mixture;
II.c glyceryl mono- and/or di- and/or triesters of at least one fatty acid;
II.d mixtures of glyceryl monoesters, diesters and triesters of at least one fatty acid;
II.e and mixtures thereof.

Even more preferably, compound II is chosen from the following group of products: hydrogenated cotton seed oil, hydrogenated soybean oil, hydrogenated palm oil, glyceryl behenate, hydrogenated castor oil, tristearine, tripalmitine, trimyristine, yellow wax, hard fatty substance or fat useful as suppository bases, anhydrous dairy fats, lanolin, glyceryl palmitostearate, glyceryl stearate, lauryl macrogol glycerides, cetyl alcohol, polyglyceryl diisostearate, diethylene glycol monostearate, ethylene glycol monostearate, Omega 3 and any mixture thereof,

preferably from the following subgroup of products: hydrogenated cotton seed oil, hydrogenated soybean oil, hydrogenated palm oil, glyceryl behenate, hydrogenated castor oil, tristearine, tripalmitine, trimyristine and any mixture thereof.

In practice, and without this being limiting, it is preferable for compound II to be chosen:
from the group of products sold under the following brand names: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol diisostéarique®, Geleol®, Hydrine®, Monthyle® and mixtures thereof;
and also from the group of additives whose codes are as follows: E 901, E 907, E 903 and mixtures thereof;
and preferably from the group of products sold under the following brand names: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab® and mixtures thereof.

According to another advantageous characteristic of the invention, the coating or the matrix allowing the controlled release of AP are free of talc.

Advantageously, the coating or the matrix of the microparticles may comprise, besides the essential constituents I and II, other standard ingredients known to those skilled in the art, especially such as:
dyes,
plasticizers, for instance dibutyl sebacate,
hydrophilic compounds, for instance cellulose and derivatives thereof or polyvinylpyrrolidone and derivatives thereof,
and mixtures thereof.

Without this being limiting and according to an even more preferred embodiment, the coating of the coated controlled-release microparticles of AP comprises only one composite coating film I-II.

Other details and examples of compositions and processes for obtaining the microparticles according to the second embodiment of the invention are given in WO-A-03/030 878, the content of which is incorporated into the present specification by reference.

In quantitative terms, the coating monolayer may represent, for example, not more than 40% and preferably not more than 30% by weight of the microparticles. Such a limited content of coating makes it possible to produce dosage units each containing a high dose of AP, without exceeding a size that is prohibitive with regard to swallowing. The compliance with the treatment and thus the success of the treatment are undoubtedly improved thereby.

According to a third embodiment, the oral pharmaceutical form comprises at least two populations of microparticles. Each population of controlled-release AP microparticles may be in accordance with the first or the second embodiment presented above.

According to one variant -2i- of the second embodiment combined with the third embodiment, the oral pharmaceutical form implemented in the use according to the invention comprises at least two populations of microparticles with different dissolution profiles, for at least 1 pH value of between 1.4 and 7.4.

According to one variant -2ii- of the second embodiment combined with the third embodiment, the oral pharmaceutical form implemented in the use according to the invention comprises at least two populations of controlled-release AP microparticles that differ in their respective initiating pH values.

According to yet another variant -2iii- of the second embodiment combined with the third embodiment, the oral pharmaceutical form according to the invention comprises at least two populations of controlled-release AP microparticles that differ in their respective initiation times.

According to a fourth embodiment, the oral pharmaceutical form implemented in the use according to the invention comprises at least one population of controlled-release AP microparticles and at least one population of immediate-release AP microgranules.

According to one variant -2iv- of the second embodiment combined with the fourth embodiment, the oral pharmaceutical form implemented in the use according to the invention comprises:
at least one population of immediate-release AP microgranules;
at least one population P1 of controlled-release AP microparticles, and
at least one population P2 of controlled-release AP microparticles;
   and, moreover, the respective initiating pH values of P1 and of P2 differ by at least 0.5 pH unit, preferably by at least 0.8 pH unit and even more preferentially by at least 0.9 pH unit.

Advantageously, the respective initiating pH values of the various populations of controlled-release AP microparticles are between 5 and 7.

According to one variant -2v- of the second embodiment combined with the fourth embodiment, the oral pharmaceutical form implemented in the use according to the invention comprises:
at least one population of immediate-release AP microgranules;
at least one population P1′ of controlled-release AP microparticles, the initiating pH value of which is equal to 5.5; and
at least one population P2′ of controlled-release AP microparticles, the initiating pH value of which is between 6.0 inclusive and 6.5 inclusive.

The populations P1, P2, P1′ and P2′ of the variants -2iv- and -2v- of the 2^{nd} embodiment comprise controlled-release AP microparticles, obtained in accordance with the 2^{nd} embodiment.

To illustrate the variants of the invention according to which the microunits with immediate release of AP are present in the oral pharmaceutical form implemented in the use according to the invention, it may be pointed out that these variants may correspond to the case where this pharmaceutical form comprises, for example, at least one population of immediate-release AP microparticles.

When the oral pharmaceutical form implemented in the use according to the first embodiment comprises controlled-release AP microparticles, the dissolution profiles of the said microparticles between pH 1 and pH 5 are similar, according to the similarity factor f2 calculated as indicated in the FDA directive SUPAC-MR—Modified Release Solid Oral Dosage Forms, i.e. as soon as f2≧50%.

The above-targeted coated microparticles may have several structures.

Thus, according to a first form of structure of the coated microparticles, at least some of the controlled-release AP microparticles of the oral pharmaceutical form each comprise:
a core containing AP and
at least one coating covering the core and allowing the controlled release of the said AP.

According to a second form of structure of the coated microparticles, at least some of the said controlled-release AP microparticles of the oral pharmaceutical form each comprise:
a core comprising:
   a neutral core, and
   at least one active layer comprising the AP and coating the neutral core,
and at least one coating covering the core and allowing the controlled release of the AP.

Advantageously, the proportion of AP in the microunits (expressed as weight % of solids relative to the total mass of the microunits) is between 5 and 80, preferably between 10 and 75 and even more preferentially between 15 and 70.

According to one possibility, the immediate-release AP microgranules are non-coated nuclei of controlled-release AP microparticles.

As regards the preparation of the coated microparticles according to the invention, this relates to microencapsulation techniques that are accessible to those skilled in the art, the principles of which are summarized in the article by C. Duverney and J. P. Benoit in “L'actualité chimique”, December 1986. More specifically, the technique under consideration is microencapsulation by film coating, leading to individualized “reservoir” systems as opposed to matrix systems.

For further details, reference will be made to patent EP-B-0 953 359.

The AP particles whose granulometry is desired and necessary for producing the microparticles according to the invention may be pure AP crystals and/or crystals that have undergone a pre-treatment via one of the conventional techniques of the art, for instance granulation, in the presence of at least one standard binder and/or an agent for modifying the intrinsic solubility characteristics of the AP. The AP may be, for example, deposited on the core via techniques known to those skilled in the art, for instance the “spray coating” technique in a fluidized air bed or formed by wet granulation, compacting, extrusion-spheronization, etc.

Advantageously, the oral pharmaceutical form implemented in the use according to the invention is in a single daily oral dose form comprising from 1000 to 500 000 microunits containing AP.

More specifically, the oral pharmaceutical form implemented in the use according to the invention may be in the form of a daily oral single dose comprising from 1000 to 500 000 controlled-release AP microparticles.

The oral pharmaceutical form according to the invention may be provided in the form of a sachet of powder of controlled-release AP microparticles, a liquid suspension or a suspension to be reconstituted of controlled-release AP microparticles, a tablet possibly containing controlled-release AP microparticles, or a gel capsule containing controlled-release AP microparticles.

A subject of the present invention is also the oral pharmaceutical form as described above in the context of the use according to the invention and taken as such independently of the use according to the present invention.

According to another of its objects, the invention is directed towards the use of controlled-release AP microparticles as defined above and optionally immediate-release AP microgranules as defined above, for the preparation of microparticulate pharmaceutical or dietetic oral dosage forms, preferably in the form of tablets that are advantageously orodispersible, powders or gel capsules.

According to another of its objects, the invention is directed towards the use of controlled-release AP microparticles and/or microgranules as defined above and optionally immediate-release AP microgranules as defined above, for the preparation of a therapeutically safe microparticulate oral pharmaceutical form, designed such that once the said pharmaceutical form has been ingested, the microparticles it comprises are dispersed and individualized when they reach the stomach, which allows these microparticles to be subjected to regular and gradual gastric emptying, whether the patient is fed or fasted during the dosage intake, thus ensuring release of the AP in its gastrointestinal absorption window, which may participate towards reducing the variability of the plasmatic concentration profiles of AP.

According to yet another of its subjects, the invention is directed towards coated microparticles and/or matrix microgranules per se as defined above.

According to other subjects thereof, the invention is directed towards:
a therapeutic treatment method characterized in that it consists in administering, preferably as a single daily oral dose, the pharmaceutical form implemented in the use according to the invention as defined above;
a reproducible method for human or animal oral therapeutic treatment, characterized in that it consists essentially in administering orally a pharmaceutical form comprising AP contained in a coated form or in a matrix and allowing the controlled release of the said AP, such that this form administered orally to a sample of human individuals leads, irrespective of the fed or fasted state of the individuals, to a reduction in the inter- and/or intra-individual standard deviation of the Cmax and/or of the Tmax, which makes it possible to ensure lower variability of the efficacy and of the therapeutic safety of the pharmaceutical form, compared with an immediate-release AP pharmaceutical form administered to this same sample of individuals, at the same dose;
a reproducible method for human or animal oral therapeutic treatment, characterized in that it consists essentially in administering orally a pharmaceutical form comprising AP contained in a coating or in a matrix that imparts to this pharmaceutical form properties such that the oral administration of this pharmaceutical form, in the fed state, to a sample of individuals, leads to a reduction in the number or to the disappearance of the individual plasmatic concentration profiles having a Tmax of less than or equal to one hour and preferably less than or equal to 1.5 hours, to the benefit of the individual plasmatic concentration profiles having a Tmax of greater than one hour and preferably greater than 1.5 hours, which makes it possible to ensure lower variability of the efficacy and of the therapeutic safety of the pharmaceutical form, compared with an immediate-release AP pharmaceutical form administered to this same sample of individuals;
a reproducible method for human or animal oral therapeutic treatment, characterized in that it consists essentially in administering orally a pharmaceutical form comprising AP contained in a coated form or in a matrix, to reduce the variability of the plasmatic profiles during the oral administration of this pharmaceutical form to a sample of individuals, compared with an immediate-release AP pharmaceutical form administered to this same sample of individuals, which makes it possible to ensure lower variability of the efficacy and of the therapeutic safety of the pharmaceutical form;
a reproducible method for human or animal oral therapeutic treatment, characterized in that it consists essentially in administering orally a pharmaceutical form comprising AP whose solubility is dependent on the gastric pH, the AP being contained in a coated form or in a matrix that impart to this pharmaceutical form properties such that the oral administration of this pharmaceutical form to a sample of individuals leads to a reduction in the coefficient of inter- and/or intra-individual variation of the Tmax, compared with an immediate-release AP pharmaceutical form administered to this same sample of individuals, at the same dose, which makes it possible to ensure lower variability of the efficacy and of the therapeutic safety of the pharmaceutical form.

### EXAMPLES

### Example 1

### Solubility of Irbesartan as a Function of the pH

The solubility of Irbesartan varies greatly in the gastric pH range:

| | Solubility at 37° C. |
|---|---|
| pH | (mg/l) |
| 1.2 | 2420 |
| 2.0 | 362 |
| 3.0 | 48 |
| 4.5 | 20 |
| 5.5 | 65 |
| 6.8 | 666 |

### Example 2

### Preparation of a Gel Capsule Containing a 300 mg Dose of Irbesartan

### Step 1:

700 g of Irbesartan and 100 g of Klucel EF® (hydroxypropylcellulose/Aqualon) are dispersed in 3000 g of isopropanol. The suspension is sprayed onto 200 g of neutral microspheres (Asahi-Kasei) in a Glatt GPCG1 spray coater.

The granulate obtained has an Irbesartan concentration of 70%.

### Step 2:

70 g of ethylcellulose (Ethocel 20 Premium/Dow), 10 g of Plasdone K29/32® (Povidone/ISP), 5 g of Cremophor RH 40 (macrogol glyceryl hydroxystearate/BASF) and 15 g of castor oil are dispersed in a mixture composed of 60% isopropanol and 40% acetone. This solution is sprayed onto 900 g of Irbesartan granulate (prepared in Step 1).

The microparticles obtained are then placed in a size 0 el gelatin capsule. The dose of Irbesartan per gel capsule was set in this test at 300 mg (i.e. 476 mg of microparticles). This gel capsule constitutes the final form of the medicament.

### Example 3

### Solubility of Eprosartan as a Function of the pH

The solubility of Eprosartan varies greatly in the gastric pH range:

| | Solubility at 37° C. |
|---|---|
| pH | (mg/l) |
| 1.2 | 795 |
| 2.0 | 142 |
| 3.0 | 33 |
| 4.5 | 18 |
| 5.5 | 15 |
| 6.8 | 1191 |

### Example 4

### Preparation of a Gel Capsule Containing a 300 mg Dose of Eprosartan

### Step 1:

950 g of Eprosartan and 30 g of Povidone (Plasdone K29/32) are dry-premixed in the tank of a high-shear granulator (Aeromatic PMA1) for 5 minutes. This pulverulent mixture is then granulated with water (200 g). The granules are dried at 40° C. in a ventilated oven, and then calibrated on a 500 μm screen. The 200-500 μm fraction is selected by screening. The granulate obtained has an Eprosartan concentration of 95%.

### Step 2:

65 g of ethylcellulose (Ethocel 7 Premium/Dow), 15 g of Plasdone K29/32® (Povidone/ISP), 10 g of Lutrol F-68 (Poloxamer 188/BASF) and 10 g of triethyl citrate are dispersed in a mixture composed of 60% isopropanol and 40% acetone. This solution is sprayed onto 900 g of Eprosartan granulate (prepared in Step 1).

The microparticles obtained are then placed in a size 1 el gelatin capsule. The dose of Eprosartan per gel capsule was set in this test at 300 mg (i.e. 350 mg of microparticles). This gel capsule constitutes the final form of the medicament.

### Example 5

### Solubility of Acyclovir as a Function of the pH

The solubility of Acyclovir varies greatly in the gastric pH range:

| | Solubility at 37° C. |
|---|---|
| pH | (mg/l) |
| 1.2 | 10500 |
| 2.0 | 6000 |
| 2.5 | 3300 |
| 4.5 | 2600 |
| 6.8 | 2500 |

### Example 6

### Preparation of a Tablet Containing a 200 mg Dose of Acyclovir

### Step 1:

800 g of Acyclovir, 100 g of lactose, 50 g of Povidone (Plasdone K29/32) and 50 g of magnesium stearate are dry-premixed in a wheel mixer. Tablets composed of 250 mg of the above mixture are prepared using a Korsch tableting press.

### Step 2:

30 g of ethylcellulose (ethylcellulose N7/Aqualon), 8 g of Klucel EF2® (hydroxypropylcellulose/Aqualon), 7 g of Lutrol F-68 (Poloxamer 188/BASF) and 5 g of dibutyl sebacate are dispersed in a mixture composed of 70% ethanol and 30% water. This solution is sprayed onto 950 g of Acyclovir tablets (prepared in Step 1).

These coated tablets constitute the final form of the medicament.

### Example 7

### Preparation of a Gel Capsule Containing a 200 mg Dose of Acyclovir

### Step 1:

970 g of Acyclovir and 30 g of Plasdone K29/32® (Povidone/ISP) are dry-premixed in the tank of a high-shear granulator (Aeromatic PMA1) for 5 minutes. This pulverulent mixture is then granulated with water (200 g). The granules are dried at 40° C. in a ventilated oven and then calibrated on a 500 μm screen. The 200-500 μm fraction is selected by screening.

The granulate obtained has an Acyclovir concentration of 97%.

### Step 2:

50 g of ethylcellulose (Ethocel 20 Premium/Dow), 18 g of Plasdone K29/32®(Povidone/ISP), 6 g of Lutrol F-68 (Poloxamer 188/BASF) and 6 g of castor oil are dispersed in a mixture composed of 70% ethanol and 30% water. This solution is sprayed onto 920 g of Acyclovir granulate (prepared in Step 1) in a Glatt GPCG1.

The microparticles obtained are then placed in a size 1 gelatin capsule. The dose of Acyclovir per gel capsule was set in this test at 200 mg (i.e. 302 mg of microparticles). This gel capsule constitutes the final form of the medicament.

### Example 8

### In Vivo Data

### Pharmaceutical Forms Implemented:

C1: Zovirax® 200 mg tablet (commercial reference)
M1: Gel capsule of microcapsules according to Example 6, containing 600 mg of Acyclovir

### Description of the Test:

3 C1 tablets and one M1 gel capsule are administered after dinner to 48 healthy volunteers in the course of a crossed-test study. The plasmatic concentrations of Acyclovir are measured at times: 0-0.5-1-2-3-4-6-10-12-16-20-24-36-48 hours post-administration.

### Pharmacokinetic Results:

The main pharmacokinetic parameters obtained are as follows:

| Product | C24h (ng/ml) | Cmax (ng/ml) | Standard deviation Cmax (ng/ml) |
|---|---|---|---|
| M1 | 23.2 | 618.7 | 185.7 |
| C1 | 22.1 | 975.1 | 208.3 |

It is found that the M1 form according to the invention allows the Cmax/C24h ratio to be reduced by a factor of 1.65 compared with the C1 form. At the same time, the variability of the Cmax is reduced by a factor of 1.12.

1. Use,
in an oral pharmaceutical form comprising at least one AP whose solubility varies by a factor of at least 3, preferably at least 10 and even more preferentially at least 30 under gastric pH conditions of between 1.0 and 5.5 and preferably between 1.5 and 5.0,
of a coating or of a matrix including the said AP and allowing the controlled release of the said AP,
in order for this form administered orally to a sample of individuals to lead, irrespective of the fed or fasted state of the individuals, to a reduction in the inter- and/or intra-individual standard deviation of the Cmax and/or of the Tmax,
which makes it possible to ensure a lower variability of the efficacy and of the therapeutic safety of the pharmaceutical form,
relative to an immediate-release AP pharmaceutical form administered to this same sample of individuals, at the same dose.

2. Use
of AP contained in a coated form or in a matrix allowing the controlled release of this AP, whose solubility varies by a factor of at least 3, preferably at least 10 and even more preferentially at least 30 under gastric pH conditions of between 1.0 and 5.5 and preferably between 1.5 and 5.0,
for the manufacture of an oral pharmaceutical form which, after oral administration to a sample of individuals, leads, irrespective of the fed or fasted state of the individuals, to a reduction in the inter- and/or intra-individual standard deviation of the Cmax and/or of the Tmax,
which makes it possible to ensure a lower variability of the efficacy and of the therapeutic safety of the pharmaceutical form,
relative to an immediate-release AP pharmaceutical form administered to this same sample of individuals, at the same dose.

3. Use according to claim 1 or 2, characterized in that the pharmaceutical form is defined by a number of daily dosage intakes that is identical to that of an immediate-release pharmaceutical form comprising the same dose of AP.

4. Use according to at least one of the preceding claims, characterized in that the AP is chosen from the group comprising the following families of AP:
proton pump inhibitors, angiotensin II receptor antagonists [or ARB (Angiotensin Receptor Blocker)], antiulcer agents, antidiabetic agents, anticoagulants, antithrombotic agents, hypolipidaemiants, antiarrhythmic agents, vasodilators, antiangina agents, antihypertensives, vasoprotective agents, fertility promoters, labour inducers and inhibiters, contraceptives, antibiotics, antifungal agents, antiviral agents, anticancer agents, anti-inflammatory agents, analgesics, antiepileptics, antiparkinson agents, neuroleptic agents, hypnotic agents, anxiolytic agents, psychostimulators, antimigraine agents, antidepressants, antitussif agents, antihistamine or antiallergic agents, agents for combating congestive heart insufficiency, angina pectoris, left ventricular hypertrophy, cardiac arrhythmia, myocardial infarction, reflex tachycardia, ischaemic heart disease, atheromatosis, hypertension associated with diabetes mellitus, portal hypertension, vertigo, bradycardia, arterial hypotension, water retention, acute renal insufficiency, orthostatic hypotension and cerebral congestion, and combinations of all the products mentioned above taken within one or more families;
ARBs being preferred, and more particularly the ARBs selected from the subgroup comprising:
Irbesartan, Olmesartan, Eprosartan, Candesartan, Candesartan Cilexetil, Valsartan, Telmisartan, Zolasartin and Tasosartan, taken alone or as mutual mixtures.

5. Use according to at least one of the preceding claims, characterized in that the pharmaceutical form contains a dose D of AP and in that the placing of this pharmaceutical form in contact with 100 ml of water at 37° C. in ambient atmosphere does not allow, at equilibrium, the total dissolution of the dose D for at least one gastric pH value of between 1 and 5.5.

6. Use according to at least one of the preceding claims, characterized in that the inter-individual standard deviation reduction factor (f) of the Cmax is defined as follows:
f≧1.05; preferably f≧1.5 and even more preferentially f is between 2.0 and 20.

7. Use according to at least one of the preceding claims, characterized in that the coating or the matrix of the pharmaceutical form are designed such that they allow the controlled release of the AP, firstly to prevent any premature and/or massive and/or rapid release of the AP and subsequently any deleterious plasmatic overconcentration of AP, and secondly to ensure therapeutic cover between two dosage intakes.

8. Use according to at least one of the preceding claims, characterized in that the coating or the matrix of the pharmaceutical form is designed such that the oral administration of this form, to a sample of individuals, leads to a mean peak/trough modulation of the plasmatic profiles of the AP, less than the mean peak/trough modulation for the AP of the same sample of individuals who received the same dose of an immediate-release AP form.

9. Use according to claim 8, characterized in that the peak/trough modulation reduction factor (g) is such that: g≧1.05; preferably g≧1.5 and even more preferentially g is between 2.5 and 20.

10. Use according to at least one of the preceding claims, characterized in that the coating or the matrix of the pharmaceutical form are designed such that the oral administration of this form, to a sample of individuals, leads to variability of the peak/trough modulation of the plasmatic profiles of the AP metabolite, less than the variability of the peak/trough modulation of the AP metabolite, for the same sample of individuals who received the same dose of an immediate-release AP form.

11. Use according to claim 10, characterized in that the factor (g′) for reduction of the standard deviation of the peak/trough modulation is such that:
g′≧1.05; preferably g′≧1.5 and even more preferentially g′ is between 2.5 and 20.

12. Use according to at least one of the preceding claims, characterized in that the oral pharmaceutical form contains AP in the form of microunits, which may be:
microparticles individually consisting of a core that comprises AP and that is coated with at least one coating allowing the controlled release of the AP;
and/or microgranules individually consisting of a matrix that includes AP and that allows the controlled release of the AP;
and/or immediate-release AP microgranules.

13. Use according to at least one of claims 1 to 9, characterized in that the oral pharmaceutical form is a tablet free of microparticles individually consisting of a core comprising AP and coated with at least one coating allowing the controlled release of the AP and/or free of microgranules individually consisting of a matrix including AP and allowing the controlled release of the AP.

14. Use according to at least one of the preceding claims, characterized in that the pharmaceutical form makes it possible to obtain, after a dosage intake, a plasmatic profile defined as follows:
Cmax/C24h≦Cmax*/C24h*
preferably 1.5×Cmax/C24h≦Cmax*/C24h*
and even more preferentially 2.0×Cmax/C24h≦Cmax*/C24h*
with
C24h representing the mean plasmatic concentration of AP 24 hours after intake,
C24h* representing the mean plasmatic concentration of AP obtained under the same conditions as C24h, with a reference immediate-release oral pharmaceutical form, containing the same dose of AP,
Cmax representing the mean maximum plasmatic concentration of AP after intake,
Cmax* representing the mean maximum plasmatic concentration of AP obtained under the same conditions as Cmax, with a reference immediate-release oral pharmaceutical form, containing the same dose of AP.

15. Use according to at least one of claims 7 to 9, characterized in that the oral pharmaceutical form comprises microparticles and has an in vitro dissolution profile such that the time t (70%) after the administration and after which 70% of the AP is released is between 1 and 24 hours, preferably between 2 and 12 hours and even more preferentially between 2 and 8 hours.

16. Use according to claim 15, characterized in that the in vitro dissolution profile of the oral pharmaceutical form is such that, for any value of the time t between 2 hours and t (70%), preferably for any value of the time t between 1 hour and t (70%), the percentage of dissolved AP is greater than or equal to 35 t/t (70%).

17. Use according to at least one of claims 7 to 9, characterized in that the oral pharmaceutical form is such that:
the release of the AP is governed by two different initiation mechanisms, one being based on a pH variation and the other allowing the release of the AP after a predetermined residence time in the stomach;
at a constant pH of 1.4, the dissolution profile comprises a lag phase lasting less than or equal to 7 hours, preferably less than or equal to 5 hours and even more preferentially between 1 and 5 hours,
and the passage from pH 1.4 to pH 7.0 leads to a release phase starting without any lag time.

18. Use according to claim 17, characterized in that the oral pharmaceutical form has a dissolution profile, measured in an in vitro dissolution test, as indicated below:
less than 20% of the AP is released after 2 hours at pH 1.4;
at least 50% of the AP is released after 16 hours at pH 1.4.

19. Use according to claim 17, characterized in that the oral pharmaceutical form comprises controlled-release AP microparticles, the initiating pH value of which is between 5.0 inclusive and 7.0 inclusive.

20. Use according to claim 19, characterized in that the oral pharmaceutical form comprises controlled-release AP microparticles, the initiating pH value of which is between 6.0 inclusive and 6.5 inclusive.

21. Use according to one of claims 7 to 9 and 15 to 20, characterized in that the oral pharmaceutical form comprises at least two populations of microparticles.

22. Use according to one of claims 7 to 9 and 15 to 21, characterized in that the oral pharmaceutical form comprises at least one population of controlled-release microparticles and/or microgranules and/or at least one population of immediate-release microgranules.

23. Use according to one of claims 7 to 9 and 18 to 22, characterized in that the oral pharmaceutical form comprises at least two populations of controlled-release microparticles and/or microgranules with different dissolution profiles, for at least one pH value of between 1.4 and 7.4.

24. Use according to one of claims 7 to 9 and 18 to 23, characterized in that the oral pharmaceutical form comprises at least two populations of controlled-release AP microparticles and/or microgranules that differ in their respective initiating pH values.

25. Use according to one of claims 7 to 9 and 18 to 24, characterized in that the oral pharmaceutical form comprises at least two populations of controlled-release AP microparticles and/or microgranules that differ in their respective initiation times.

26. Use according to one of claims 7 to 9 and 18 to 25, characterized in that the oral pharmaceutical form comprises:
at least one population of immediate-release AP microgranules;
at least one population P1 of controlled-release AP microparticles and/or microgranules, and
at least one population P2 of controlled-release AP microparticles and/or microgranules;
and in that the respective initiating pH values of P1 and of P2 differ by at least 0.5 pH unit, preferably by at least 0.8 pH unit and even more preferentially by at least 0.9 pH unit.

27. Use according to one of claims 7 to 9 and 18 to 26, characterized in that the respective initiating pH values of the various populations of controlled-release AP microparticles and/or microgranules are between 5 and 7.

28. Use according to one of claims 7 to 9 and 18 to 27, characterized in that the oral pharmaceutical form comprises:
at least one population of immediate-release AP microgranules;
at least one population P1′ of controlled-release AP microparticles and/or microgranules, the initiating pH value of which is equal to 5.5; and
at least one population P2′ of controlled-release AP microparticles and/or microgranules, the initiating pH value of which is between 6.0 inclusive and 6.5 inclusive.

29. Use according to one of claims 7 to 28, characterized in that the oral pharmaceutical form comprises at least one population of immediate-release AP microgranules whose behaviour in an in vitro dissolution test is such that at least 80% of the AP is released in 1 hour at any pH between 1.4 and 7.4.

30. Use according to one of claims 7 to 29, characterized in that the oral pharmaceutical form is in a single daily oral dose form comprising from 1000 to 500 000 microunits containing AP.

31. Use according to one of claims 7 to 30, characterized in that the oral pharmaceutical form is in the form of a daily oral single dose comprising from 1000 to 500 000 controlled-release AP microparticles and/or microgranules.

32. Use according to one of the preceding claims, characterized in that the oral pharmaceutical form is in the form of a sachet of powder, a liquid suspension or a suspension to be reconstituted, a tablet or a gel capsule.

33. Use according to one of the preceding claims, characterized in that the pharmaceutical form comprises at least one active principle AP other than the AP.

34. Use according to at least one of claims 7 to 9 and 15 to 17, characterized in that the pharmaceutical form comprises controlled-release AP microparticles and/or microgranules, the composition of the coating or of the matrix of which is chosen from the group comprising formula A or formula B described below:
Formula A
A1—at least one film-forming polymer (P1) that is insoluble in the fluids of the gastro intestinal tract, present in a proportion of from 50 to 90 and preferably 50 to 80 by weight of solids relative to the total mass of the coating composition and especially comprising at least one water-insoluble cellulose derivative;
A2—at least one nitrogenous polymer (P2) present in a proportion of from 2% to 25% and preferably 5% to 15% by weight of solids relative to the total mass of the coating composition and consisting of at least one polyacrylamide and/or one poly-N-vinylamide and/or one poly-N-vinyl lactam;
A3—at least one plasticizer present in a proportion of from 2% to 20% and preferably from 4% to 15% by weight of solids relative to the total mass of the coating composition and consisting of at least one of the following compounds: glycerol esters, phthalates, citrates, sebacates, cetyl alcohol esters, castor oil;
A4—at least one surfactant and/or lubricant, present in a proportion of from 2% to 20% and preferably from 4% to 15% by weight of solids relative to the total mass of the coating composition and chosen from anionic surfactants and/or from nonionic surfactants and/or from lubricants; the said agent possibly comprising only one or a mixture of the abovementioned products;
or
Formula B
B1—at least one film-forming polymer that is insoluble in the fluids of the gastrointestinal tract,
B2—at least one water-soluble polymer,
B3—at least one plasticizer,
B4—and optionally at least one surfactant/lubricant preferably consisting of at least one anionic surfactant and/or at least one non ionic surfactant.

35. Use according to at least one of claims 7 to 34, characterized in that the controlled-release AP microparticles and/or microgranules have a mean diameter (Dm in μm) of less than 1000, preferably between 50 and 800 and even more preferentially between 50 and 500.
